(19) United States
(12) Patent Application Publication
(10) Pub. No.: US 2013/0327679 A1
(43) Pub. Date: Dec. 12, 2013

(54) SURGICAL KITS AND METHODS

(71) Applicant: Edward S. Holt, Annapolis, MD (US)

(72) Inventor: Edward S. Holt, Annapolis, MD (US)

(21) Appl. No.: 13/490,251

(22) Filed: Jun. 6, 2012

(51) Int. Cl. A61L 2/04; A61J 1/00; B65B 55/12

(52) U.S. Cl. 206/570; 53/428; 422/1

(57) ABSTRACT

Disclosed are surgical kits and methods of making a surgical device for proximation of body parts. An exemplary method comprises receiving a ferrule defining a ferrule axis, the ferrule being received a room temperature; sterilizing the ferrule received in the previous step; receiving a sleeve defining a sleeve axis. The sleeve includes a projection configured to exert a force, parallel to the sleeve axis, against the ferrule, the sleeve being received at room temperature; and sterilizing the sleeve.

### BACKGROUND OF THE INVENTION

1. Field of the Invention

This invention relates generally to surgical kits and methods and, more particularly, to surgical kits and methods for resisting a tensile force in order to maintain body parts in proximity.

2. Description of Related Art

An orthopedic prosthetic device can rely on a flexible tensioned element to maintain approximation of two or more body parts, such as bones. The flexible tensioned element can be made of metal cable or cordage that is a composite of a tough cover, such as polyester, and a core with high tensile strength and low creep, such as an Aramid fiber. For example, an orthopedic implant can include a metal flange connected to a cord at one end, the cord passing through a bone and then through or around a second bone and tied to a second metal flange thereby holding the two bones together.

The effectiveness of such a device may be limited by the wearing out of the cord over time.

### SUMMARY OF THE INVENTION

To address the problem above, there is a method of making a surgical device for proximation of body parts. The method comprises receiving a ferrule defining a ferrule axis, the ferrule being received a room temperature; sterilizing the ferrule received in the previous step; receiving a sleeve and defining a sleeve axis, the sleeve including a projection configured to exert a force, parallel to the sleeve axis, against the ferrule, the sleeve being received at room temperature; and sterilizing the sleeve.

According to another aspect of the present invention, there is a surgical kit, to enable proximation of body parts in a surgical procedure. The surgical kit comprises a ferrule defining a ferrule axis, the ferrule being sterile; a first package enclosing the ferrule, the first package defining a sterile interior; a sleeve and defining a sleeve axis, the sleeve including a projection configured to exert a force, parallel to the sleeve axis, against the ferrule, the sleeve sterile; a second package enclosing the sleeve, the second package defining a sterile interior; and a third package enclosing the first and second packages.

### BRIEF DESCRIPTION OF THE DRAWINGS

References are made to the following text taken in connection with the accompanying drawings, in which:

FIG. 1 shows a combination of elements in accordance with a first exemplary embodiment of the present invention.

FIG. 2 shows a process performed in accordance with the first exemplary embodiment.

FIG. 3 shows a subsequent phase of the process.

FIG. 4 shows another process.

FIG. 5 shows a subsequent phase of the other process.

FIG. 6 shows a subsequent phase.

FIG. 7 shows a subsequent phase.

FIG. 8 shows a subsequent phase.

FIG. 9 is a diagram emphasizing a sub process.

FIG. 10 is a diagram showing a subsequent phase of the process.

FIG. 11 is a cross section view emphasizing certain aspects of the configuration shown in FIG. 10.

FIG. 12 shows another process.

FIG. 13 shows a subsequent phase of the other process.

FIG. 14 shows a subsequent phase.

FIG. 15 shows a subsequent phase.

FIG. 16 shows a subsequent phase.

FIG. 17 is a diagram showing a subsequent phase of the process.

FIG. 18 is a cross section view emphasizing certain aspects of a configuration.

FIG. 19 is a cross section view of the configuration shown in 18 at a subsequent time point.

FIG. 20 shows another process.

FIG. 21 shows a subsequent phase of the other process.

FIG. 22 shows a subsequent phase.

FIG. 23 shows a subsequent phase.

FIG. 24 shows a subsequent phase.

FIG. 25 is a diagram showing a subsequent phase of the process.

FIG. 26 is a diagram emphasizing certain aspects of the configuration shown in FIG. 25.

The accompanying drawings which are incorporated in and which constitute a part of this specification, illustrate embodiments of the invention and, together with the description, explain the principles of the invention, and additional advantages thereof. Certain drawings are not necessarily to scale, and certain features may be shown larger than relative actual size to facilitate a more clear description of those features. Throughout the drawings, corresponding elements are labeled with corresponding reference numbers.

### DETAILED DESCRIPTION OF EXEMPLARY EMBODIMENTS

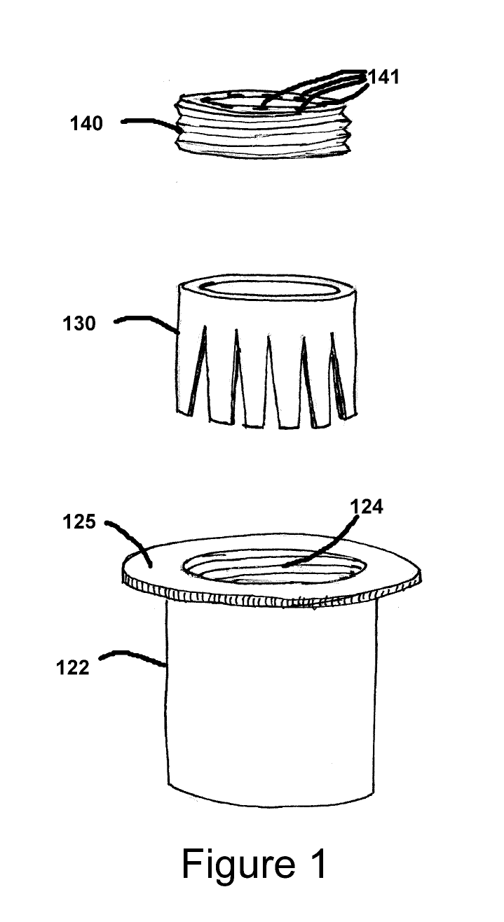

FIG. 1 shows a sleeve 122 in accordance with a first exemplary embodiment of the present invention. The sleeve 122 defines internal threads 124 and a flange 126.

The ferrule 130 has a plurality of tapered segments. The ferrule 130 is configured to fit into the sleeve 122.

The set screw 140 defines external threats 142 that mate with internal threads 124 of the sleeve 122. The set screw 140 defines a plurality of indents 144 for engagement with a screwdriver, as described in more detail later in this disclosure.

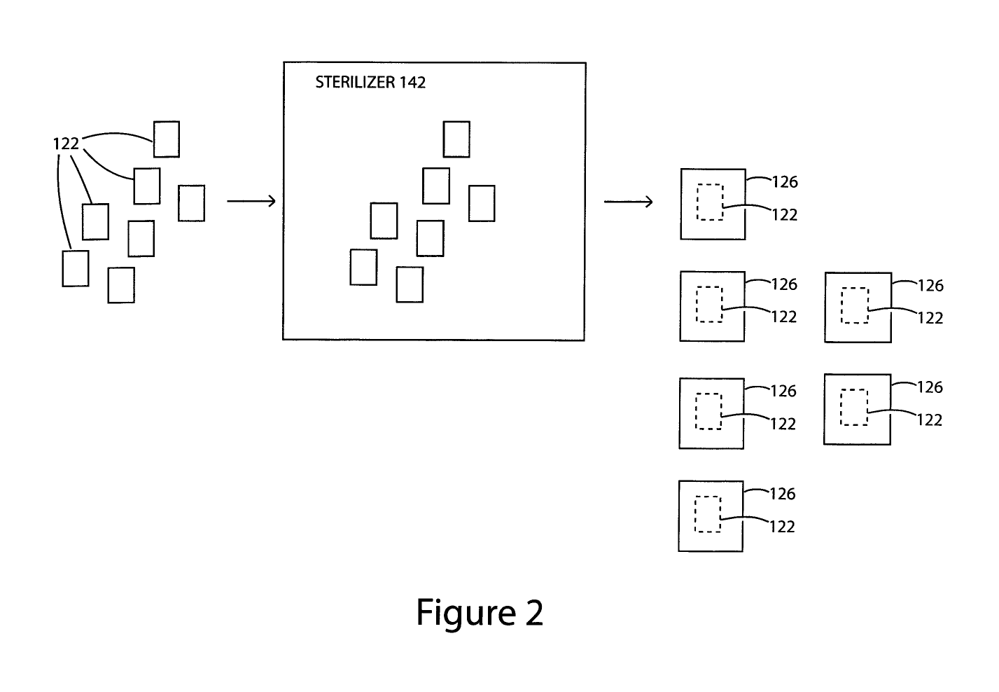

FIG. 2 shows a process performed in a factory building in accordance with the first exemplary embodiment. A plurality of sleeves 122 are placed into a sterilizer 142. The sterilizer 142 could include an oven that heats to a temperature of 100 degrees Celsius or more, in order to sterilize the sleeves 122. Subsequently, each sleeve 122 is placed in a respective sterile envelope 126 and the envelope 126 is then hermetically sealed, such that the sleeve 122 is sterile in a sterile interior of the envelope 126. Each envelope 126 contains one and only one sleeve 122.

A plurality of the ferrules 130 are placed into a sterilizer 142 and heated to a temperature of 100 degrees Celsius or more, in order to sterilize the ferrules 130. Subsequently, each ferrule 130 is placed in a respective sterile envelope 136 and the envelope 136 is then sealed hermetically sealed, such that the ferrule 136 is sterile in a sterile interior of the envelope 136. Each envelope 136 contains one and only one of the ferrules 130.

A plurality of set screws 140 are placed into a sterilizer 142 and heated to a temperature of 100 degrees Celsius or more, in order to sterilize the set screws 140. Subsequently, each set screw 140 is placed in a respective sterile envelope 146 and the envelope 146 is then sealed hermetically sealed, such that the set screw 140 is sterile in a sterile interior of the envelope 146. Each envelope 146 contains one and only one set screw 140

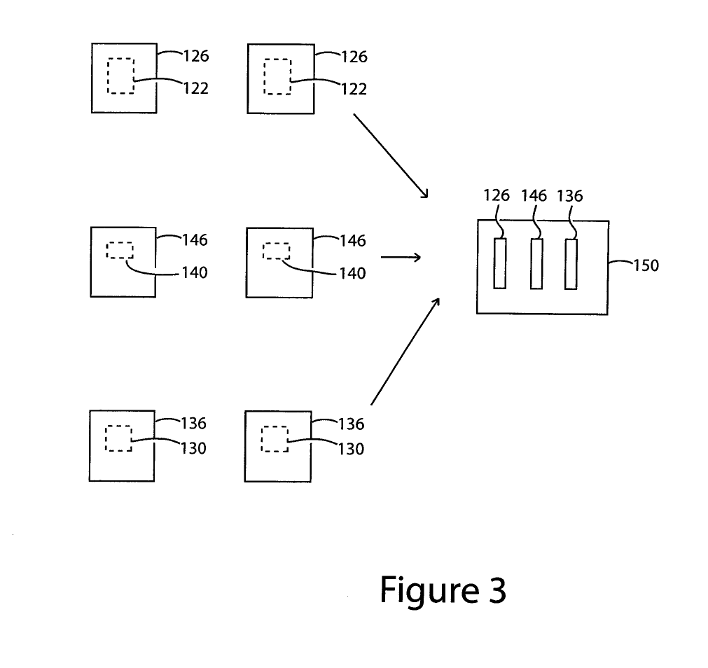

FIG. 3 shows another process performed in the factory building. An envelope 126, an envelope 146, and an envelope 136 are placed in a common box 150. The box 150 has one and only one envelope 126, one and only one envelope 146, and one and only one envelope 136.

The sterilizer 142 could include a mechanism configured to apply a chemical, physical, and/or irradiation method. Examples of chemical methods include exposure to ethylene oxide or hydrogen peroxide vapor. Examples of physical methods include sterilization by heat. Examples of irradiation methods include gamma irradiation, electron beam irradiation, and microwave irradiation.

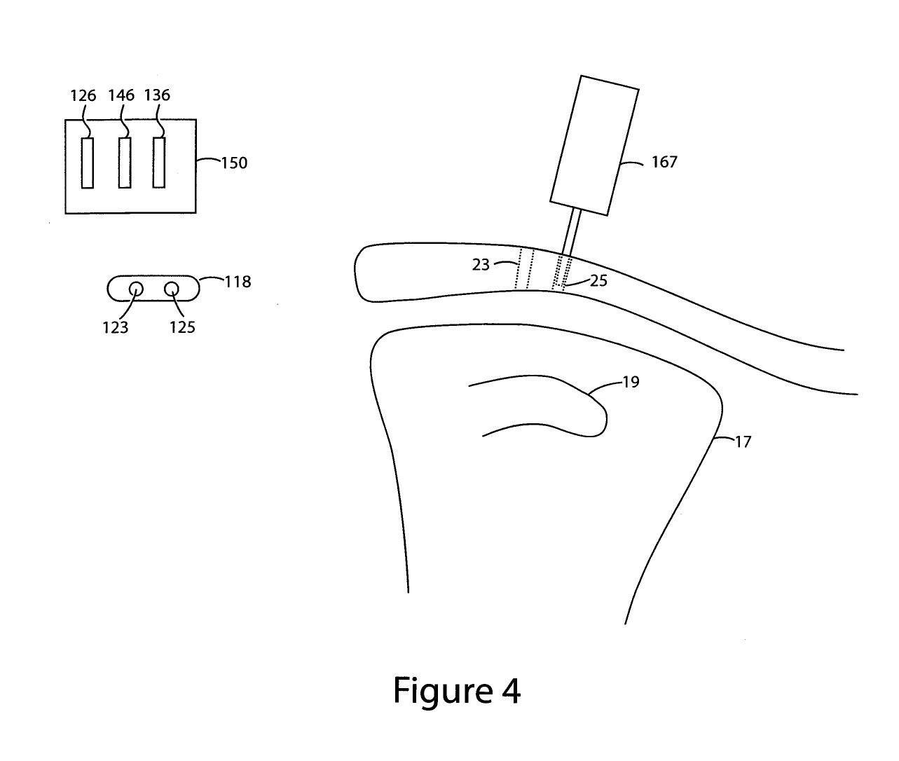

FIG. 4 shows a process performed outside of the factory building. An electric drill 167 is applied to a clavicle 21 in order to make a through-hole 23. Subsequently, the electric drill 167 is applied to the clavicle 21 in order to make a through-hole 25.

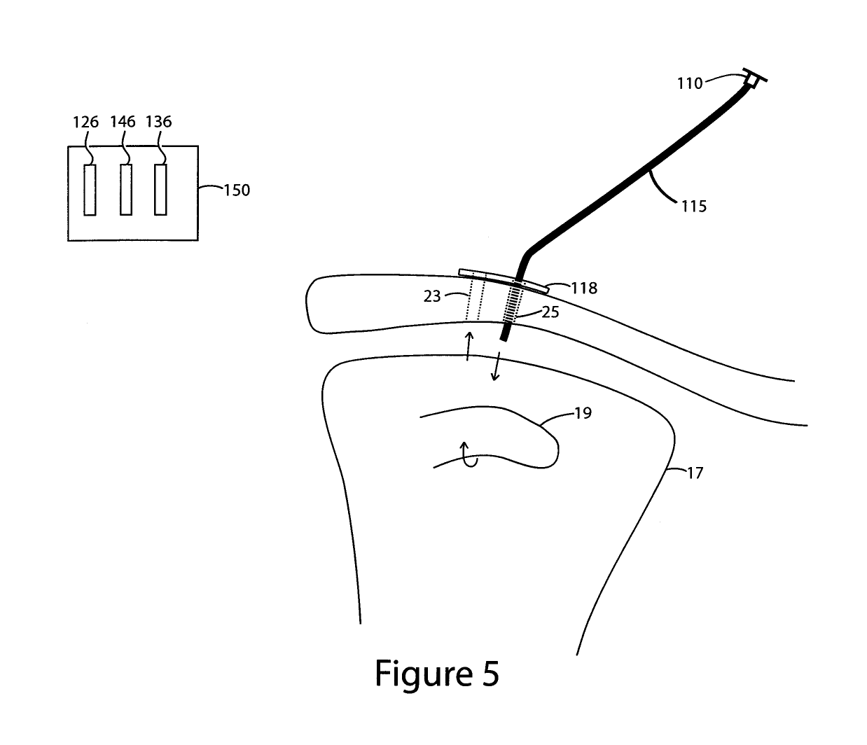

As shown in FIG. 5, a flange 118 is placed over the clavicle 21 such that a hole 123 of the flange 118 is aligned with the through-hole 23 of the clavicle 21 and a hole 125 of flange 118 is aligned with the through-hole 25 of clavicle 21. A cord 115 with fixed end stop 110 is passed through the through- the hole 125 and the hole 25, then around a coracoid process 19 of a scapula 17, and then through the through-hole 23 and the hole 123.

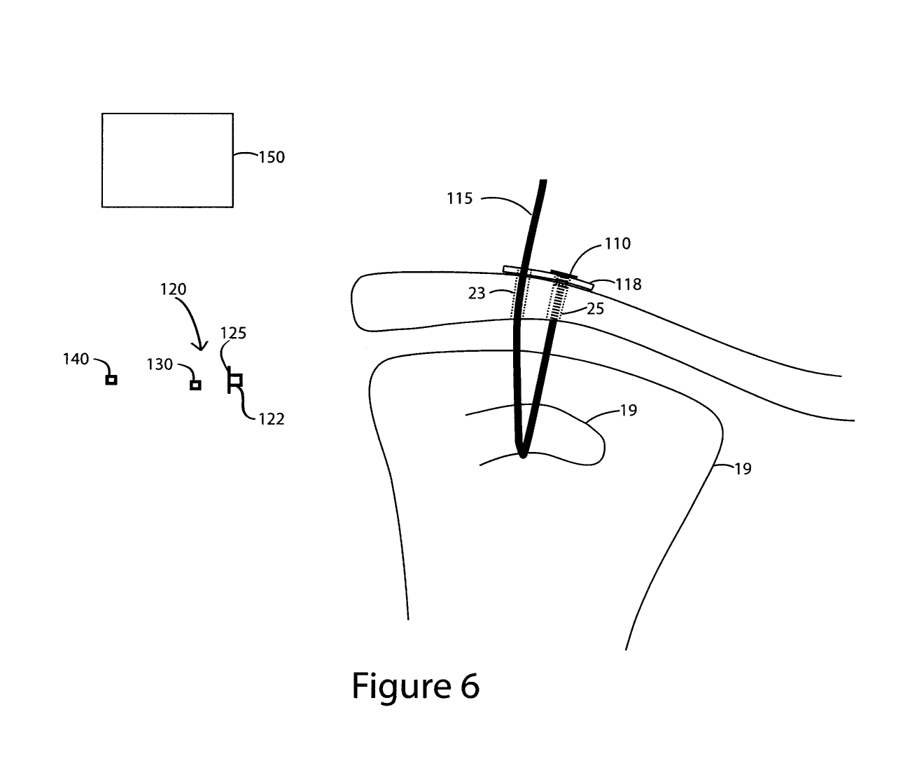

As shown in FIG. 6, the envelope 126 is removed from the interior of the box 150 and the sleeve 122 removed from the sterile interior of the envelope 126. The envelope 126 is then discarded. The envelope 146 is removed from the interior of box 150 and set crew 140 removed from the sterile interior of the envelope 146. The envelope 146 is then discarded. The envelope 136 is removed from the interior of the box 150 and the ferrule 130 removed from the sterile interior of envelope 136. The envelope 136 is then discarded.

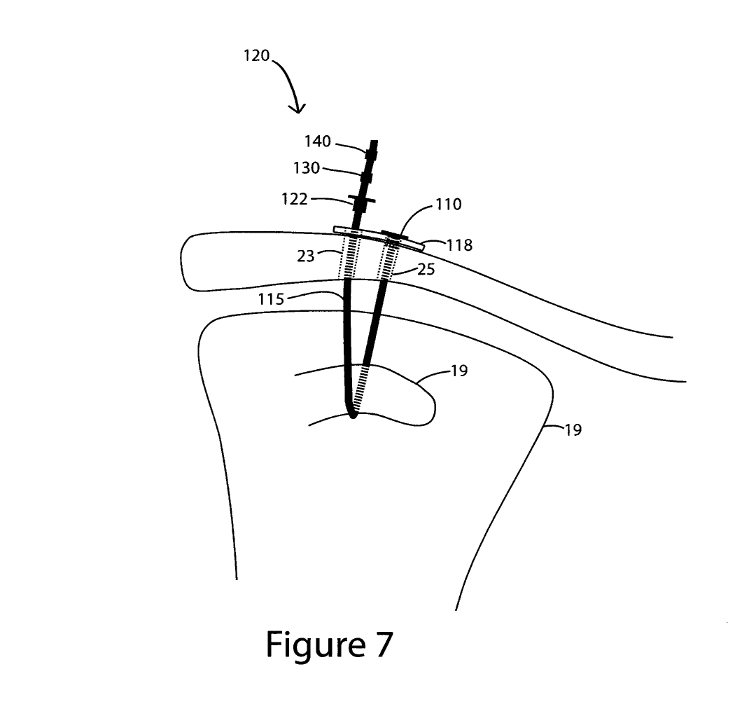

As shown in FIG. 7, the cord 115 is passed through the sleeve 122, through the ferrule 130, and though the set screw 140. Once the end of cord 115 has exited the through-hole 23 in the clavicle 21, sufficient traction is applied to the cord 115 so as to seat trailing flange assembly 110 against the flange 118. With the sleeve 122 seated against the flange 118, traction is applied to the cord 115, so as to restore alignment of the clavicle 21.

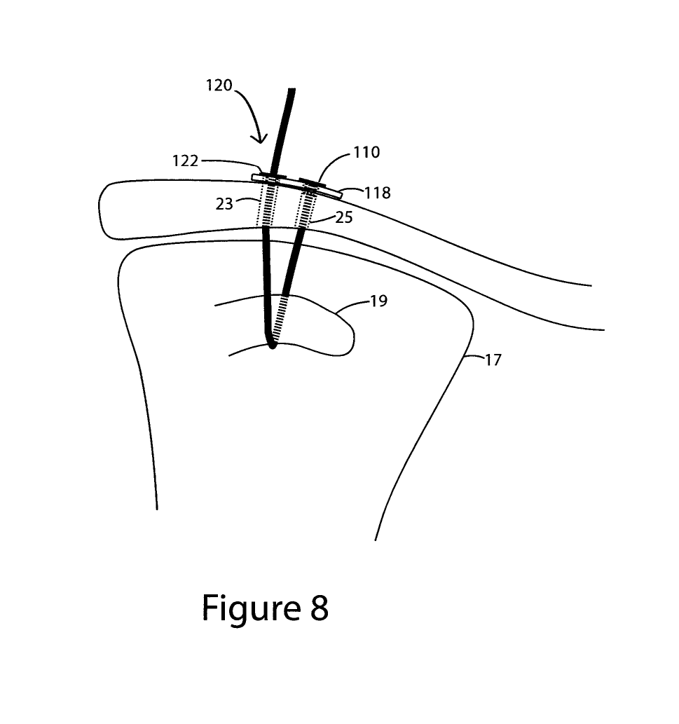

Pressure is applied to the ferrule 130 via the set screw 140, to cause the ferrule 130 to grip the cord 115 and to lock the ferrule 130 in a fixed position relative to the sleeve 122 as shown in FIG. 8.

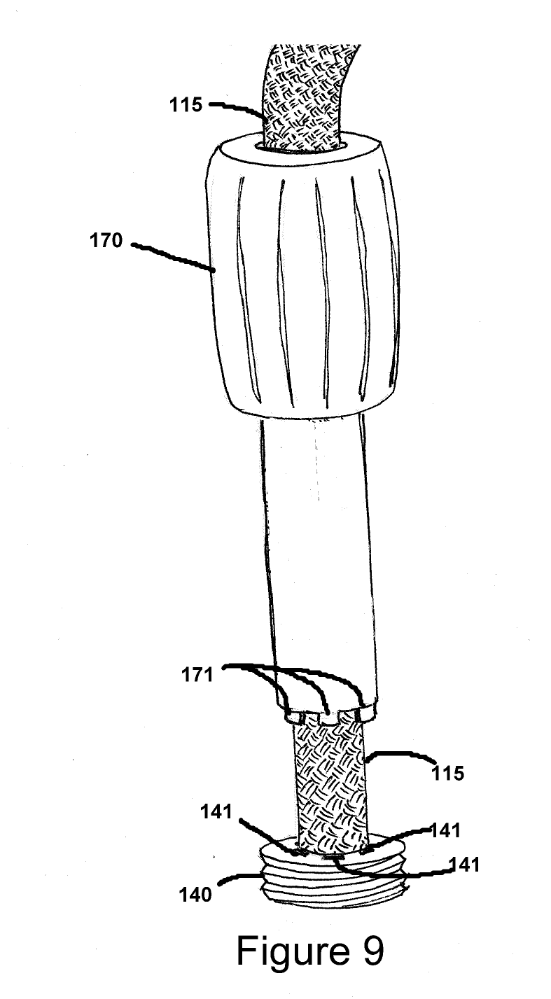

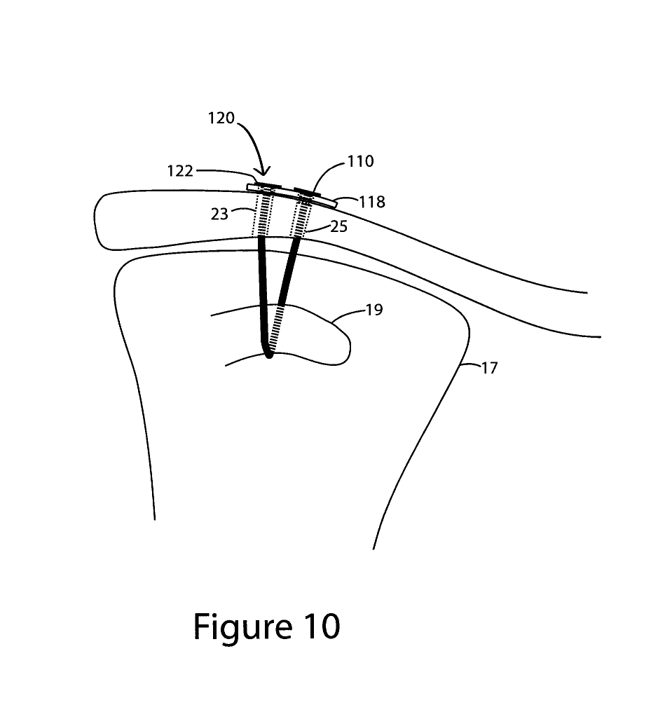

FIG. 9 shows a tool applying a turning force to the set screw 140 in order to apply pressure to the ferrule 130. A cannulated (hollow) screwdriver 170 includes teeth 171. The teeth 171 engage with the detents 141 of the set screw 140. As shown in FIG. 10, the cord 115 is then cut flush with the surface of the adjustable and stop.

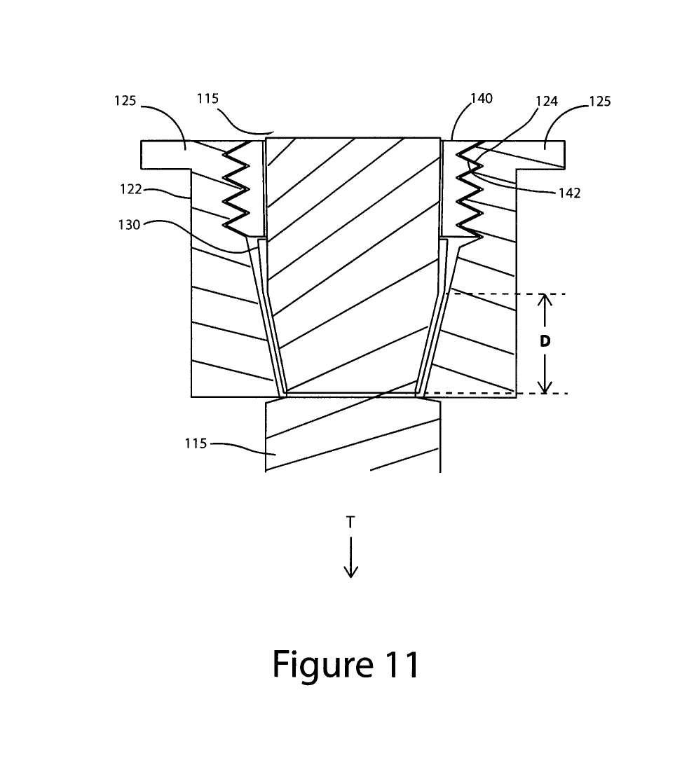

FIG. 11 is a diagram emphasizing certain aspects of the configuration shown in FIG. 10. The external threads 142 of the set screw 140 engage internal threads 124 of sleeve 122, causing the set screw 140 to exert a force downward, in the orientation of FIG. 11, onto the ferrule 130.

The tapered portions of the sleeve 122 act as an inclined plane relative to the downward force on the ferrule 130, causing the ferrule 130 to exert a compressive force inward towards the axis of the cord 115. The compressive force is applied across a distance D. The distance D exceeds the diameter of the cord 115. In other words, the distance D is more than 100 percent of the diameter of cord 115. For example, if the cord the 115 has a diameter of 1 millimeter, the distance D is greater than 1 millimeter.

The cord 115 has a diameter in the range of 1 to 10 millimeters

Because of the compression along the distance D, the cord 115 is held in place within the sleeve 122, despite a tensile force key exerted by bone and tissue.

In summary, the box 150 contains a surgical kit including a hermitically sealed package 126 having a sterile interior, a sterile sleeve 122 in the interior of the package 126. The sleeve is configured to receive the ferrule 130 and the ferrule is configured to receive the cord 115, such that the cord 115 extends from the sleeve 122 along the sleeve axis, and extends from the ferrule 130 along the ferrule axis. A section of the internal threads 124, of sleeve 122, is effectively a projection configured to exert a force, parallel to the sleeve 122 axis, against the ferrule 130, via the set screw 140.

The pressure applied via the set screw 140 causes the ferrule 130 to grip the cord 115 along a certain minimum length D of the cord, and lock the ferrule 130 in a fixed position relative to the sleeve 122.

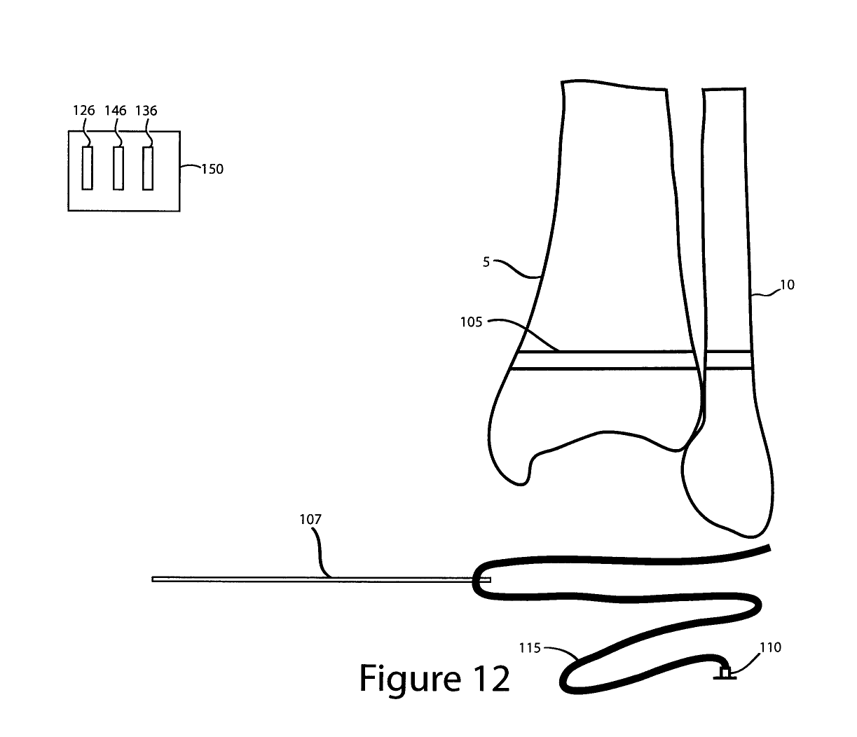

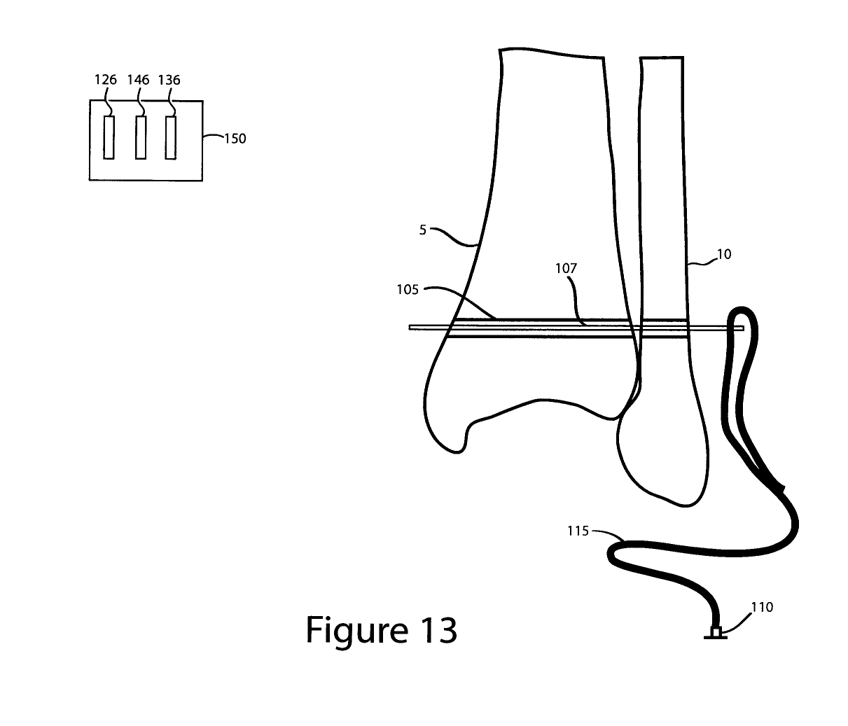

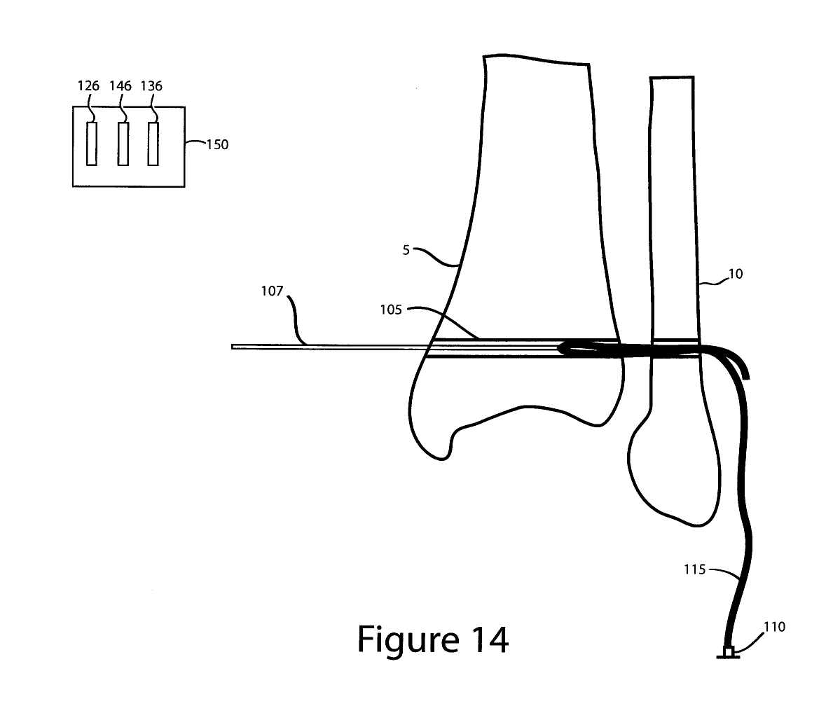

FIGS. 12-14 show a process performed outside of the factory building. A long straight needle 107 engages the cord 115 and is passed through the through-hole 105 and out the intact medial skin.

Once the needle 107 has exited the medial tibia 5, sufficient traction is applied to the cord 115 so as to seat the trailing flange assembly 110 against the fibula 10.

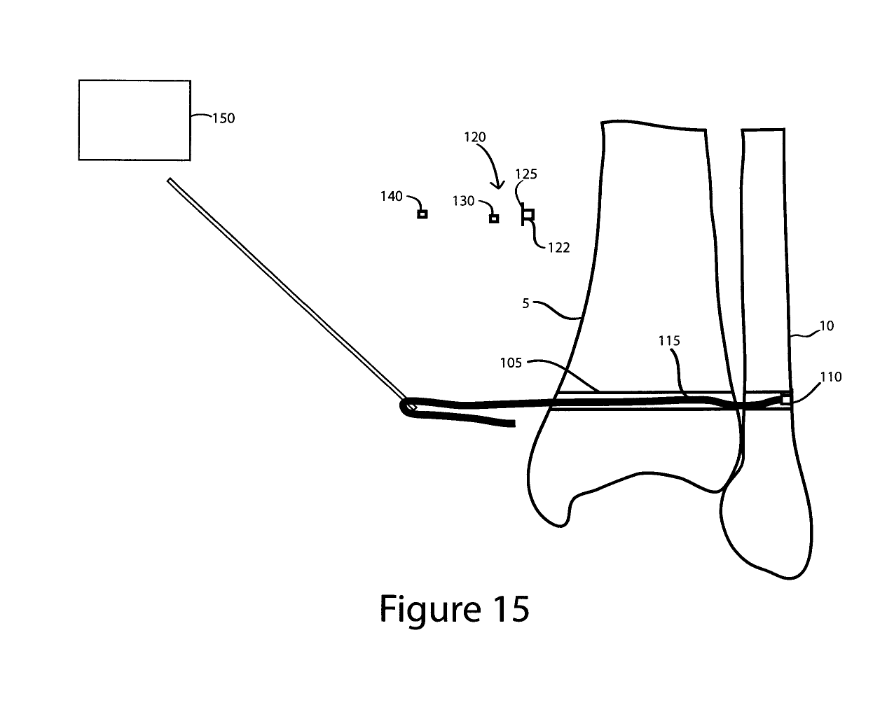

As shown in FIG. 15, the envelope 126 is removed from the interior of box 150 and the sleeve 122 removed from the sterile interior of envelope 126. The envelope 126 is then discarded. The envelope 146 is removed from the interior of box 150 and set crew 140 removed from the sterile interior of envelope 146. The envelope 146 is then discarded. The envelope 136 is removed from the interior of box 150 and the ferrule 130 removed from the sterile interior of envelope 136. The envelope 136 is then discarded.

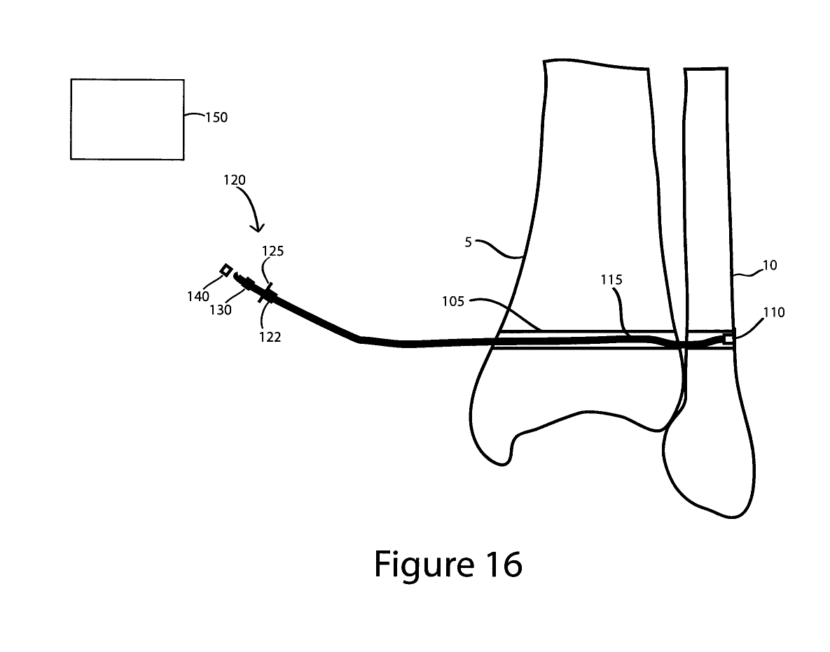

As shown in FIG. 16, the cord 115 is passed through the sleeve 122, through the ferrule 130, and though the set screw 140. The sleeve 122 is seated against the medial tibia 5. Traction is applied to the cord 115, so as to position the medial tibia 5 relative to the fibula 10.

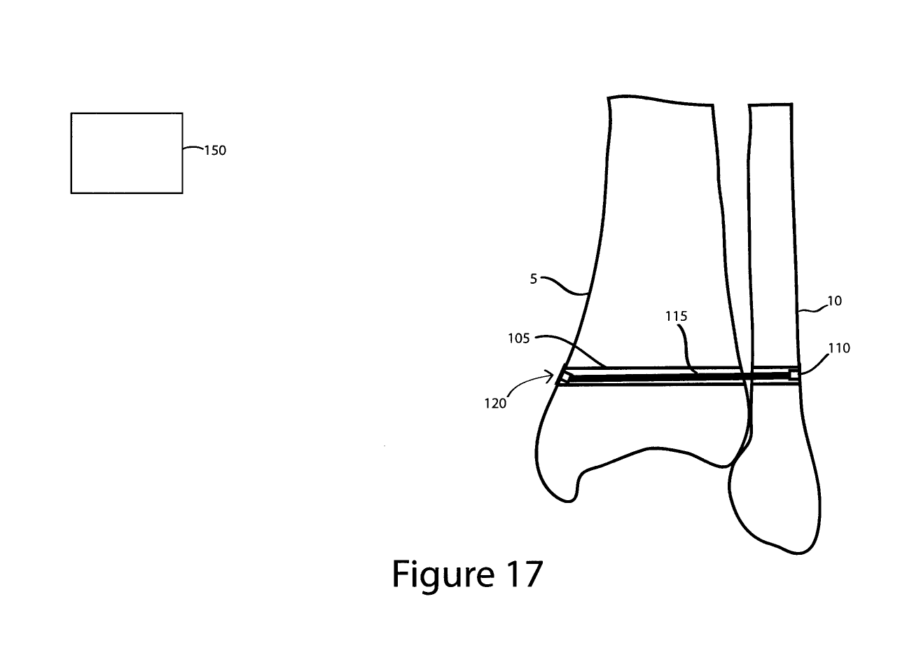

Pressure is applied to the ferrule 130 via the set screw 140, to cause the ferrule 130 to grip the cord 115 and to lock the ferrule 130 in a fixed position relative to the sleeve 122 as shown in FIG. 17.

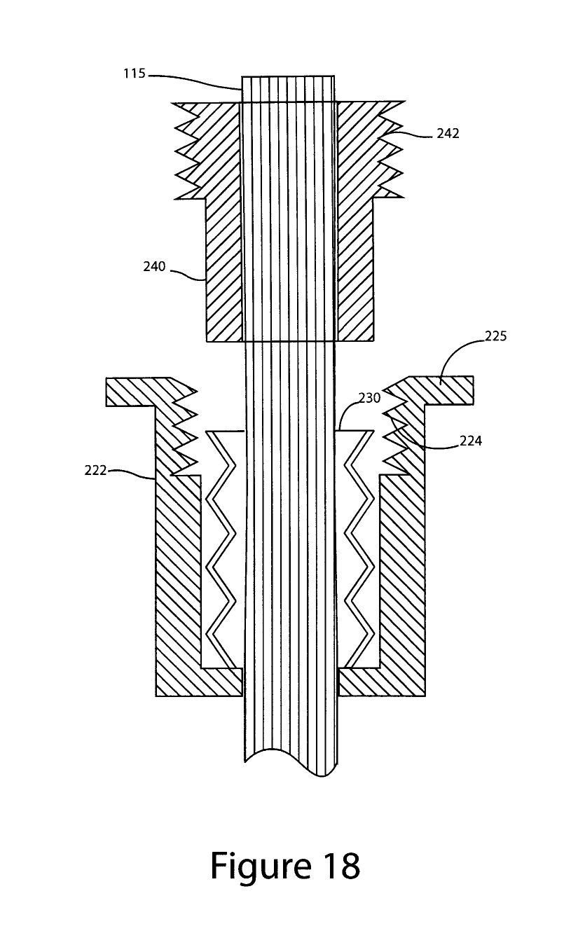

FIG. 18 shows a configuration wherein a long straight needle 107 engages the cord 115 and is passed through the through-hole 105 and out the intact medial skin.

Once the needle 107 has exited the medial tibia 5, sufficient traction is applied to the cord 115 so as to seat trailing flange assembly 110 against fibula 10.

The cord 115 is passed through the sleeve 222, through the ferrule 230, and though the set screw 240. The sleeve 222 is seated against medial tibia 5. Traction is applied to cord 115, so as to position medial tibia 5 relative to fibula 10.

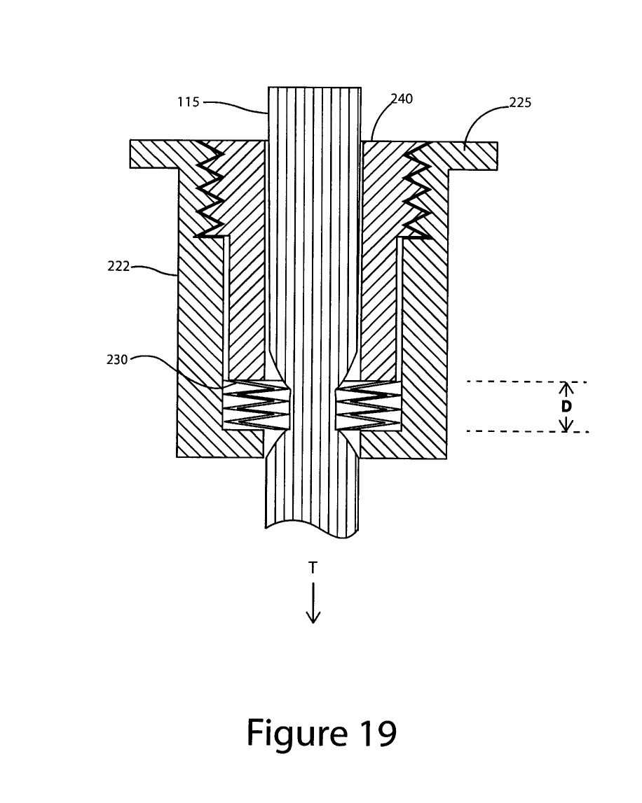

Subsequently, as shown in FIG. 19, pressure is applied to the ferrule 230 via the set screw 240, to cause the ferrule 230 to grip the cord 115 and to lock the ferrule 230 in a fixed position relative to the cord 115.

More specifically, external threads 242 of the set screw 240 engage internal threads 224 of the sleeve 222, causing the set screw 240 to exert a force downward, in the orientation of FIG. 19, onto the ferrule 230.

The downward force causes the ferrule 230 to collapse in the vertical direction, as the ferrule 230 is compressed between screw 240 and sleeve 222. The collapse of the ferrule 230 causes the ferrule 330 to expand in the horizontal direction, in the orientation of FIG. 19. Because the side of sleeve 222 is rigid, the ferrule 230 undergoes a horizontal expansion in a direction towards the axis of cord 115, causing the ferrule 230 to insert a compression inward towards the axis of cord 115. The compression force is applied across a distance D along the axis of the cord 115. The distance D exceeds the diameter of the cord 115. In other words, the distance D is more than 100 percent of the diameter of cord 115.

Because of the compression along the distance D, cord 115 is held in place within sleeve 222, despite a tensile force key exerted by bone and tissue.

To more evenly distribute the compressive force, the distance D, along the axis of the cord 115, is greater than 2 times of the diameter of cord 115.

To still more evenly distribute the compressive force, the distance D, along the axis of the cord 115, is greater than 5 times of the diameter of cord 115.

To still more evenly distribute the compressive force, the distance D, along the axis of the cord 115, is greater than 10 times of the diameter of cord 115.

In summary, the sleeve 222 is configured to receive the ferrule 230 and the ferrule 230 is configured to receive the cord 115, such that the cord 115 extends from the sleeve 222 along the sleeve axis, and extends from the ferrule 230 along the ferrule axis. A section of the internal threads 224, of sleeve 222, is effectively a projection configured to exert a force, parallel to the sleeve 222 axis, against the ferrule 230, via the set screw 240. The pressure applied via the set screw 240 causes the ferrule 230 to grip the cord 115 along a certain minimum length D of the cord, and lock the ferrule 230 in a fixed position relative to the sleeve 122 and relative to cord 115.

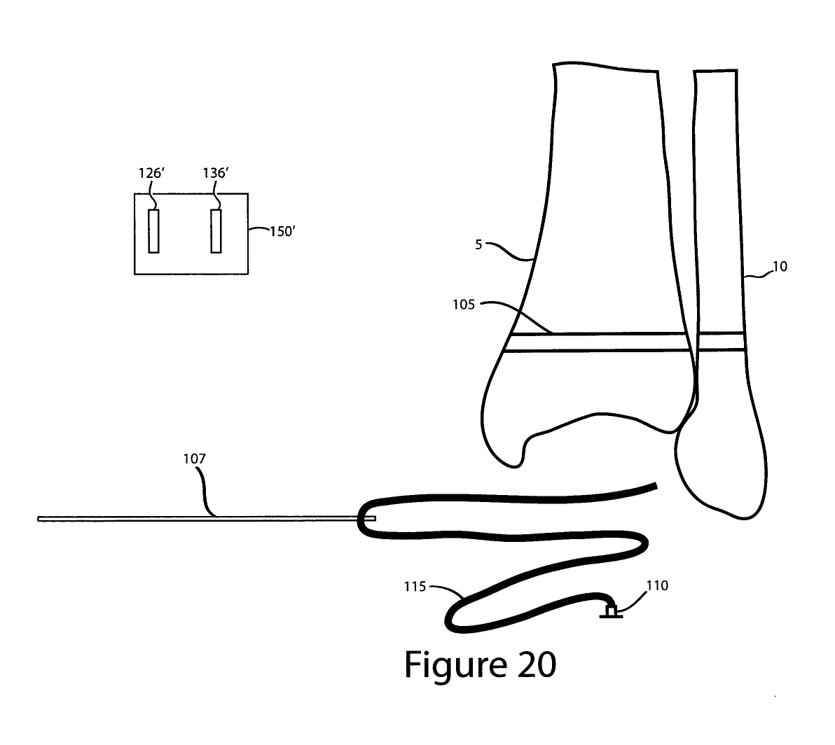

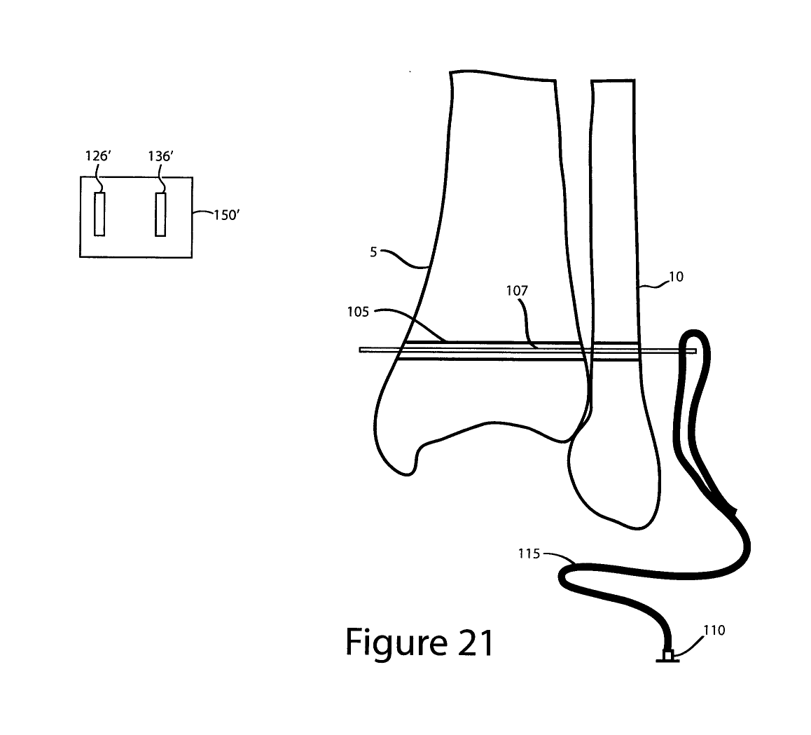

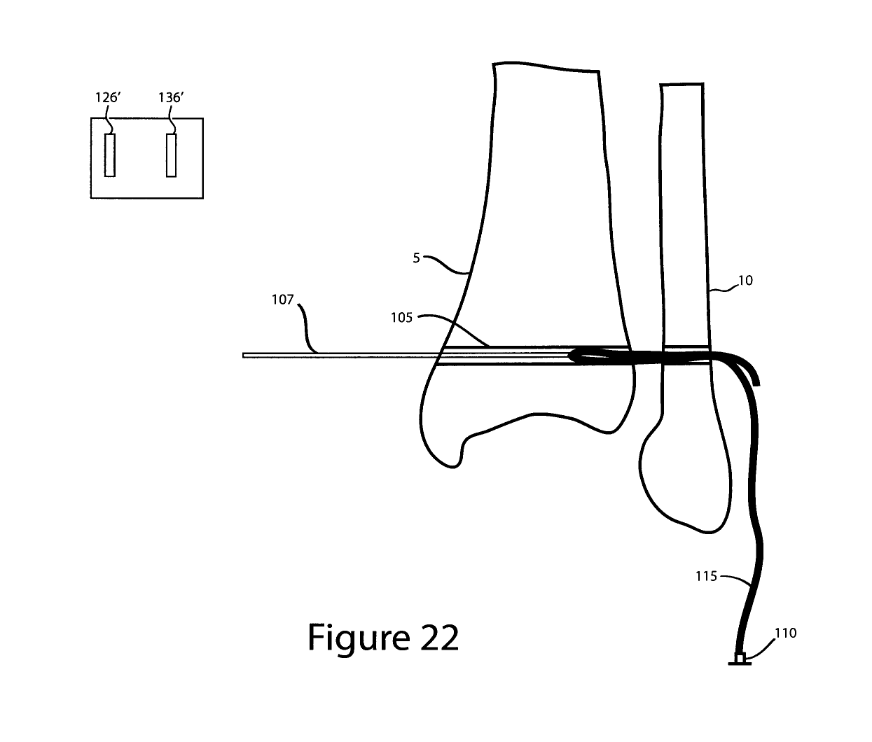

FIGS. 20-22 show a process performed outside of the factory building. A long straight needle 107 engages cord 115 and is passed through the through-hole 105 and out the intact medial skin.

Once the needle 107 has exited the medial tibia 5, sufficient traction is applied to cord 115 so as to seat trailing flange assembly 110 against fibula 10.

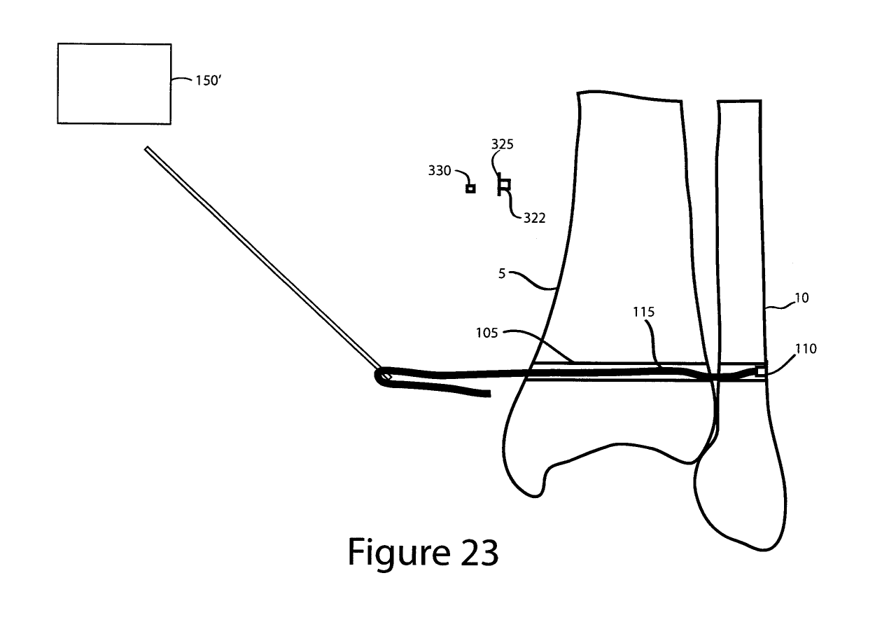

As shown in FIG. 23, envelope 126′ is removed from the interior of box 150′ and sleeve 322 removed from the sterile interior of envelope 126. Envelope 126′ is then discarded. Envelope 136′ is removed from the interior of box 150′ and the ferrule 330 removed from the sterile interior of envelope 136′. Envelope 136′ is then discarded.

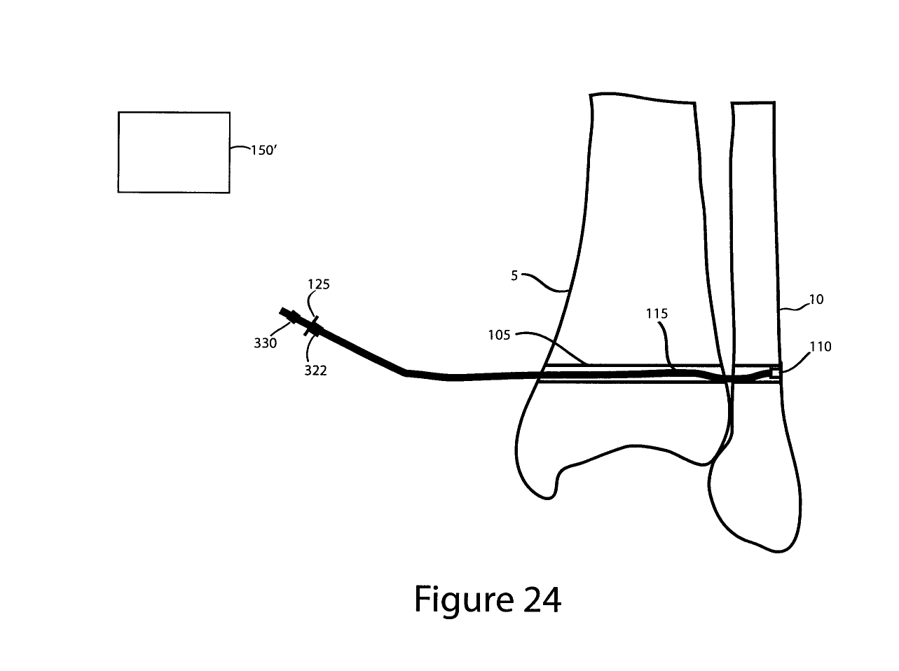

As shown in FIG. 24, the cord 115 is passed through the sleeve 322, and through the ferrule 330. The sleeve 322 is seated against medial tibia 5. Traction is applied to cord 115, so as to position medial tibia 5 relative to fibula 10.

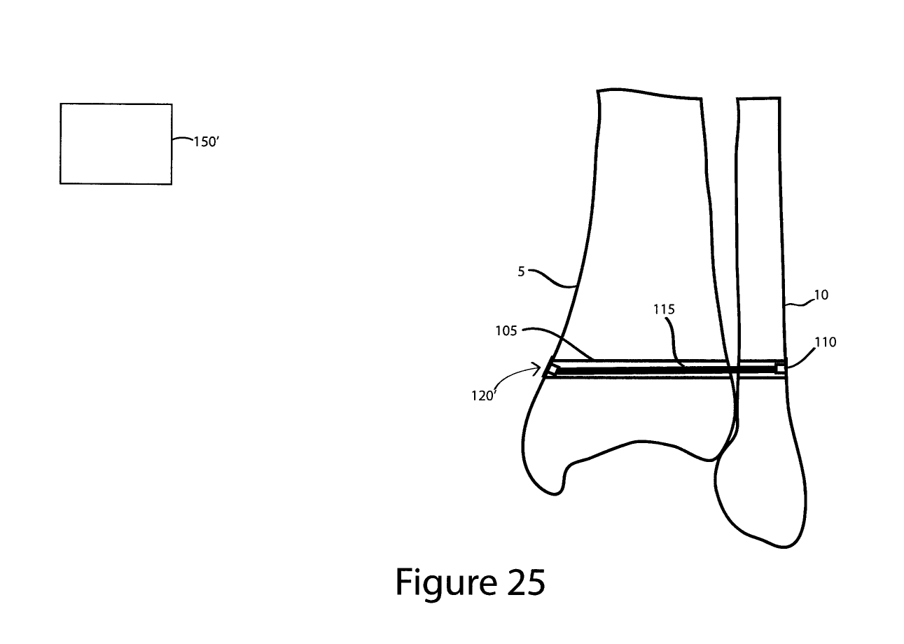

With no force applied, the ferrule 330 is wider than the aperture of sleeve 322. The surgeon inserts the narrow end of the ferrule 330 into the aperture of sleeve 322. The surgeon then exerts a pressure on the ferrule 330 along the axis of sleeve 322, causing the ferrule 330 to undergo the deformation in order to pass through the aperture of sleeve 322 such that the ferrule 330 undergoes elastic recovery (spring back), and the ferrule 330 is seated in sleeve 322, with the projection 152 of sleeve 322 exerting a pressure, on the projection 162 of the ferrule 330, along the axis of sleeve 322, to cause the ferrule 330 to grip the cord 115 and to lock the ferrule 330 in a fixed position relative to the sleeve 322 as shown in FIG. 25, where the ferrule 330-sleeve 322 combination is designated 120′.

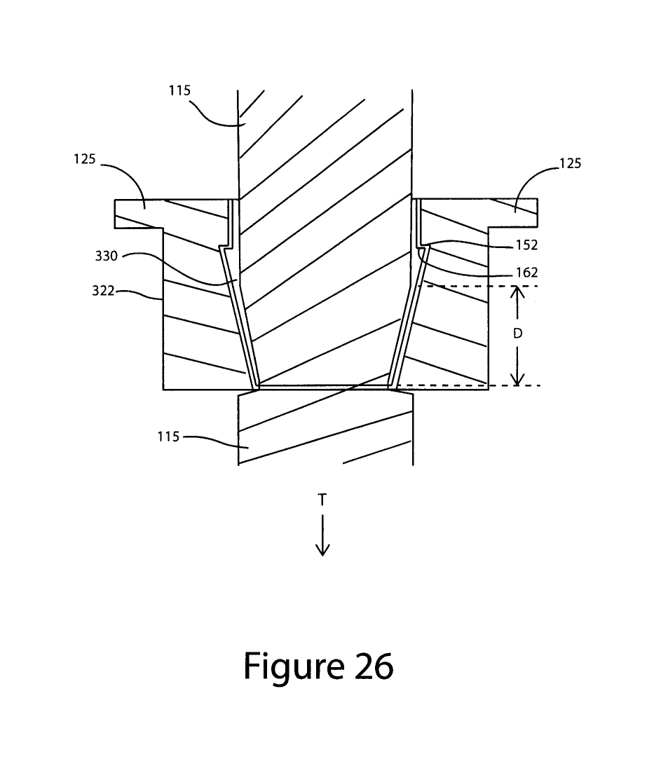

FIG. 26 is a diagram emphasizing certain aspects of the configuration shown in FIG. 25. Projection 152 of sleeve 322 engages projection 162 of the ferrule 330 such that sleeve 322 exerts a force downward, in the orientation of FIG. 26, onto the ferrule 330.

The tapered portions of the sleeve 322 act as an inclined plane relative to the downward force on the ferrule 330, causing the ferrule 330 to insert a compression inward towards the axis of cord 115. The compression force is applied across the distance D.

Because of the compression along the distance D, cord 115 is held in place within sleeve 122, despite a tensile force key exerted by bone and tissue.

In summary, box 150′ contains a surgical kit including a hermitically sealed package 126′ having a sterile interior, a sterile sleeve 322 in the interior of the package 126′. The sleeve is configured to receive the ferrule 330 and the ferrule is configured to receive the cord 115, such that the cord 322 extends from the sleeve 322 along the sleeve axis, and extends from the ferrule 330 along the ferrule axis.

Sleeve 322 includes a projection 152 configured to exert a force, parallel to the sleeve 322 axis, against the ferrule 330.

The projection 152 extends from a detent defining a ring-shaped structure in sleeve 322.

The projection 152 defines a 90 degree angle with the sleeve axis.

The components 322 and 330 undergo the same factory building sterilization and packaging processes as described in connection with the components of the first embodiment.

Benefits, other advantages, and solutions to problems have been described above with regard to specific examples. The benefits, advantages, solutions to problems, and any element(s) that may cause any benefit, advantage, or solution to occur or become more pronounced are not critical, required, or essential feature or element of any of the claims.

Additional advantages and modifications will readily occur to those skilled in the art. The invention in its broader aspects is therefore not limited to the specific details, representative apparatus, and illustrative examples shown and described. Accordingly, departures may be made from such details without departing from the spirit or the scope of Applicants' general inventive concept. The invention is defined in the following claims. In general, the words “first,” “second,” etc., employed in the claims do not necessarily denote an order.

What is claimed is:

1. A method of making a surgical device for proximation of body parts, the method comprising:
receiving a ferrule defining a ferrule axis, the ferrule being received a room temperature;
sterilizing the ferrule received in the previous step;
receiving a sleeve defining a sleeve axis, the sleeve including a projection configured to exert a force, parallel to the sleeve axis, against the ferrule, the sleeve being received at room temperature; and
sterilizing the sleeve.

2. A method of making a surgical device for proximation of body parts according to claim 1 wherein the projection is an internal thread in the sleeve, and the device further includes a set screw engaged with the internal thread, whereby the projection exerts the force against the ferrule via the set screw.

3. A method of making a surgical device for proximation of body parts according to claim 1 wherein the projection defines a ring-shaped structure.

4. A method of making a surgical device for proximation of body parts according to claim 3 wherein the projection defines a 90 degree angle with the sleeve axis.

5. A method of making a surgical device for proximation of body parts according to claim 1 wherein the sleeve defines internal threads, and the ferrule is configured to be collapsible, wherein the device further includes a set screw screwed into sleeve, the set screw defining indents configured for engagement by a cannulated screwdriver.

6. A method of making a surgical device for proximation of body parts according to claim 1 wherein the sleeve defines a tapered internal channel having a detent, and the ferrule defines a flange configured to snap into the detent of the sleeve, and defines gaps configured to close when lateral pressure is applied to the ferrule, thereby enabling the ferrule to adopt a contour of the tapered internal channel.

7. A method of making a surgical device for proximation of body parts according to claim 1 wherein the sleeve defines internal threads, and defines a tapered internal channel, and the ferrule defines gaps configured to close when lateral pressure is applied to the ferrule, thereby enabling the ferrule to adopt a contour of the tapered internal channel, wherein the device further includes a set screw screwed into sleeve.

8. A method of making a surgical kit, the method comprising the steps of claim 1 and further including the steps of
placing the sterilized sleeve in a sterile envelope; and
sealing the envelope.

9. A surgical kit, to enable proximation of body parts in a surgical procedure, the surgical kit comprising:
a ferrule defining a ferrule axis, the ferrule being sterile;
a first package enclosing the ferrule, the first package defining a sterile interior;
a sleeve defining a sleeve axis, the sleeve including a projection configured to exert a force, parallel to the sleeve axis, against the ferrule, the sleeve sterile;
a second package enclosing the sleeve, the second package defining a sterile interior; and
a third package enclosing the first and second packages.

10. A surgical kit, to enable proximate body parts in a surgical procedure, according to claim 9, wherein the first package encloses only 1 ferrule.

11. A surgical kit, to enable proximate body parts in a surgical procedure, according to claim 9, wherein the second package encloses only 1 sleeve.

12. A surgical kit, to enable proximate body parts in a surgical procedure, according to claim 9, wherein the third package encloses only 1 first package and only 1 second package.

13. A surgical kit, to enable proximate body parts in a surgical procedure, according to claim 9, further including
a cord, the cord being sterile; and
an end stop attached to the cord, the end stop being sterile.

14. A surgical kit, to enable proximate body parts in a surgical procedure, according to claim 9, further including
a cord, the cord being sterile; and
an end stop attached to the cord, the end stop being sterile,
wherein the third package encloses the cord.

15. A surgical kit, to enable proximate body parts in a surgical procedure, according to claim 9 wherein the projection is an internal thread in the sleeve, and the kit further includes a set screw engaged with the internal thread, whereby the projection exerts the force against the ferrule via the set screw.

16. A surgical kit, to enable proximate body parts in a surgical procedure, according to claim 9 wherein the projection defines a ring-shaped structure.

17. A surgical kit, to enable proximate body parts in a surgical procedure, according to claim 9 wherein the projection defines a 90 degree angle with the sleeve axis.

18. A surgical kit, to enable proximate body parts in a surgical procedure, according to claim 9 wherein the sleeve defines internal threads, and the ferrule is configured to be collapsible, wherein the device further includes a set screw screwed into sleeve, the set screw defining indents configured for engagement by a cannulated screwdriver.

19. A surgical kit, to enable proximate body parts in a surgical procedure, according to claim 9 wherein the sleeve defines a tapered internal channel having a detent, and the ferrule defines a flange configured to snap into the detent of the sleeve, and defines gaps configured to close when lateral pressure is applied to the ferrule, thereby enabling the ferrule to adopt a contour of the tapered internal channel.

20. A surgical kit, to enable proximate body parts in a surgical procedure, according to claim 9 wherein the sleeve defines internal threads, and defines a tapered internal channel, and the ferrule defines gaps configured to close when lateral pressure is applied to the ferrule, thereby enabling the ferrule to adopt a contour of the tapered internal channel, wherein the device further includes a set screw screwed into sleeve.
